(19) United States
(12) Patent Application Publication
(10) Pub. No.: US 2003/0144622 A1
(43) Pub. Date: Jul. 31, 2003

(54) Bandage for orthopaedic use

(76) Inventor: Nadia Kylberg, Lidingo (SE)

Correspondence Address: OSTROLENK FABER GERB & SOFFEN, 1180 AVENUE OF THE AMERICAS, NEW YORK, NY, 100368403

(21) Appl. No.: 10/203,330

(22) PCT Filed: Feb. 6, 2001
(86) PCT No.: PCT/SE01/00214

(30) Foreign Application Priority Data
Feb. 7, 2000 (SE) 0000373-1

(51) Int. Cl. A61F 13/00

(52) U.S. Cl. 602/58; 602/41

(57) ABSTRACT

A bandage for orthopaedic use comprises an elastic, flexible, relatively stiff supporting means (16) made in the shape of a body part to be supported. The supporting means is made of a cross-linked polythene material. In the method for fabricating such a bandage, the polythene material is heated to a predetermined moulding temperature, at which the material has a consistency suitable for moulding, supporting means with the desired shape then being made from the material.

The present invention relates to a bandage, comprising a flexible, relatively rigid supporting means in the shape of a body part to be protected, for orthopaedic use. The invention also relates to a method for fabricating such a bandage.

Support and protective bandaging of different kinds is used in e.g. medicine and sports. Support bandages in the form of braces are therefore employed for e.g. certain types of back complaints. These braces usually have a rigid, supportive design and are made of leather or stiff springs etc. brace wearers often find rigid and uncomfortable. The Swedish patent 417 269 describes a brassiere with a kind of support bandage in the form of a supporting means under each cup in order to reduce the pull on brassiere straps.

The objective of the present invention is to achieve a new, greatly improved type of bandage for a number of different orthopaedic applications. Another objective of the invention is to set forth a method for fabricating this bandage.

These objectives are achieved with a method of the kind initially cited with the features set forth in claim 1 and with a method according to claim 18.

A number of important advantages, compared to the prior art, are achieved through the use of a cross-linked polythene material for the bandage's supporting means. Thus, the bandage can withstand washing at temperatures up to 8020 C. without deformation or cracking of the supporting means. If the bandage is to be reusable, it is essential for the bandage to be washable at a temperature of 60° C. or more without sustaining damage, as a temperature this high has proved to be necessary for preventing the survival of bacteria. The plastic material used in e.g. the supporting means in the brassiere described in the cited Swedish patent document cannot withstand water temperatures greater than 40° C. At higher temperatures, the supporting means readily develops cracks, is often deformed and often loses its elasticity.

The plastic material used does not contain any softener which could cause a rash, allergy or carcinogenic changes.

According to one advantageous embodiment of the bandage according to the invention, the polythene material has a polythene composition containing a silane additive with at least one hydrolysable organic group with a high degree of compatibility with the polythene composition. The polythene material used in the present invention is previously known from the patent applications WO 90/07542 and WO 95/17463. The polythene materials described in these documents have hitherto only been used in certain kinds of electrical cables. This polythene material has now, surprisingly, been found to be particularly suitable for use in bandages of the kind to which the invention relates. In addition to the aforementioned advantages of the polythene material used, this material is permeable to air to some extent. This is an exceptionally useful property, as it enables the skin to ‘breathe’ through the material.

According to other advantageous embodiments of the bandage according to the invention, the supporting means can be perforated to further enhance the circulation of air through the material. The perforation holes can be both evenly and unevenly distributed across the surface of the supporting means.

According to another advantageous embodiment of the bandage according to the invention, the stiffness of the supporting means can be varied by varying material thickness and/or the degree of perforation. In this manner, the stiffness of the supporting means can be tailored to the needs of different applications.

According to yet another advantageous embodiment of the bandage according to the invention, the supporting means can be devised to largely enclose an extremity of the bandage wearer, thereby forming a support bandage for the said extremity. Plaster casts used for many types of fractures have shortcomings. The bandage according to the invention is an alternative without the shortcomings of a plaster cast. When the supporting means is devised with longitudinal slits or with a longitudinal groove with a specific width, the supporting means can be bent up for simple placement on the extremity in question. The resilience of the supporting means then enables it to rebound back to its original shape, thereby essentially enclosing the extremity. Alternately the supporting means can comprise at least two supporting means parts, devised for application to the body part to be supported, said supporting means parts then being interconnectable in some suitable fashion. The supporting means parts could e.g. be interconnected with some elastic material in order to achieve a bandage with some elasticity or interconnected with e.g. an external bandage made of a non-elastic material, thereby creating a stiff bandage. The supporting means parts can also be interconnected with different kinds of suitable fasteners.

According to yet another advantageous embodiment of the bandage according to the invention, the supporting means is belt-like and devised for insertion into each half of a brassiere, or garment with a brassiere section including brassiere cups, such as exercise suits and sports clothing, bathing suits, body stockings etc., covering the area from the middle of the front section of the brassiere or garment to the rear section of the brassiere or garment. The supporting means can be bent along a longitudinal line, mainly extending along the lower edge of the brassiere or garment half. When the brassiere or garment is worn, the belt-shaped supporting means part on one side of the said line provides support for the base of the breast and is supported by the other part which presses against the body under the breast of the wearer of the brassiere or garment.

This kind of brassiere therefore reduces shoulder strap loading, a particular advantage for women with strenuous work, as the load imposed by shoulder straps often leads to bad posture and shoulder, neck and back pain. In addition, shoulder straps often press on nerve plexuses, causing paresthesias and numbness in the arms and hands of brassiere wearers. Tests have shown that the brassiere according to the invention reduces the load on the shoulders by about 80%, thereby greatly reducing the load on the plexus brachialis and cervical nerve roots. Conventional brassieres, which are only intended to hold the breasts at the front, often contribute to bad posture, aggravated by pressure from shoulder straps, in women. The invention's supporting means extend towards the back area, thereby improving the posture of the brassiere or garment wearer. The brassiere or garment devised according to the invention therefore prevents poor posture.

Conventional brassieres are often uncomfortable for women who have had a breast implant because of the differing loads from the two breasts. Women who have undergone this surgery also run an increased risk of developing lymphoedema which is greatly aggravated by a heavy load on the shoulder strap of a brassiere or garment. This risk is accordingly reduced with a brassiere or garment devised according to the invention. When the supporting means extends from the front mid-section of the brassiere or garment towards the rear section, the load imposed by the breasts becomes more uniform.

According to another advantageous embodiment of the bandage according to the invention, the transverse edges of the supporting means, facing the mid-section of the brassiere or garment, run at an angle, at least a part of the width of the supporting means, in such a way that the transverse edges of the supporting means facing the mid-section of the brassiere form a gap between the supporting means in the basic shape of an inverted V.

When the brassiere or garment is devised as a bandage according to the invention, the bust as a whole receives excellent support. So this kind of brassiere or garment is very suitable for use in sports, especially contact sports such as handball, football etc., to prevent injury to fasciae in jumps and impacts by effectively holding the breasts still. Since the supporting means are cut to form a gap in the general shape of an inverted V, the movements of the diaphragm are not impeded to any great extent, i.e. breathing is not impeded. However, conventional brassieres have stiff tapes across this area which prevent unimpeded breathing. Moreover, the supporting means do not have any projecting corners which could penetrate into body fat, e.g. when the wearer bends over. The supporting means follows the contours of the body better.

According to yet another advantageous embodiment of the invention, the supporting means are located between the brassiere's or garment's lining and the outer covering. The supporting means have some give here, enabling them to smoothly follow the body's movements and facilitate rather than impede them. Since the supporting means are not completely fixed, they support and lift the bust in a positive way greatly enhancing the brassiere or garment wearer's freedom of movement.

According to another advantageous embodiment of the invention, the transverse edge of the supporting means are provided with means for ensuring correct mounting in the brassiere or garment. A correct position for the supporting means in the brassiere or garment, and therefore in relation to the wearer's body, is of the greatest importance to satisfactory function. If improperly installed, the supporting means will fail to provide the breasts with the intended support.

According to another advantageous embodiment of the bandage according to the invention, the supporting means is devised to provide a body part with protection against, blows, impacts etc. The bandage according to the invention can therefore serve as effective, convenient protection in sports, e.g. as leg pads for football players, knee pads, shoulder pads etc. for hockey players, rugby players etc.

In the method according to the invention, the polythene material is heated to a predetermined moulding temperature, normally from 90° C. to 180° C., at which the material can easily be moulded. The supporting means can accordingly be given the desired shape, e.g. adapted to the external shape of an arm when the bandage is to serve as a support bandage for a broken arm. One advantageous property of the material is its retention of the smoothness and elasticity it had before being heated to the moulding temperature after it has cooled down.

According to yet another advantageous embodiment of the method according to the invention, the polythene material is perforated before being heated to the moulding temperature. The perforations remain after the material has been heated to the moulding temperature, making it possible to fabricate the bandage's supporting means from pre-perforated polythene. This would simplify fabrication of the bandage according to the invention.

To explain the invention, exemplifying embodiments of the bandage according to the invention will now be described in greater detail, referring to the enclosed drawings in which

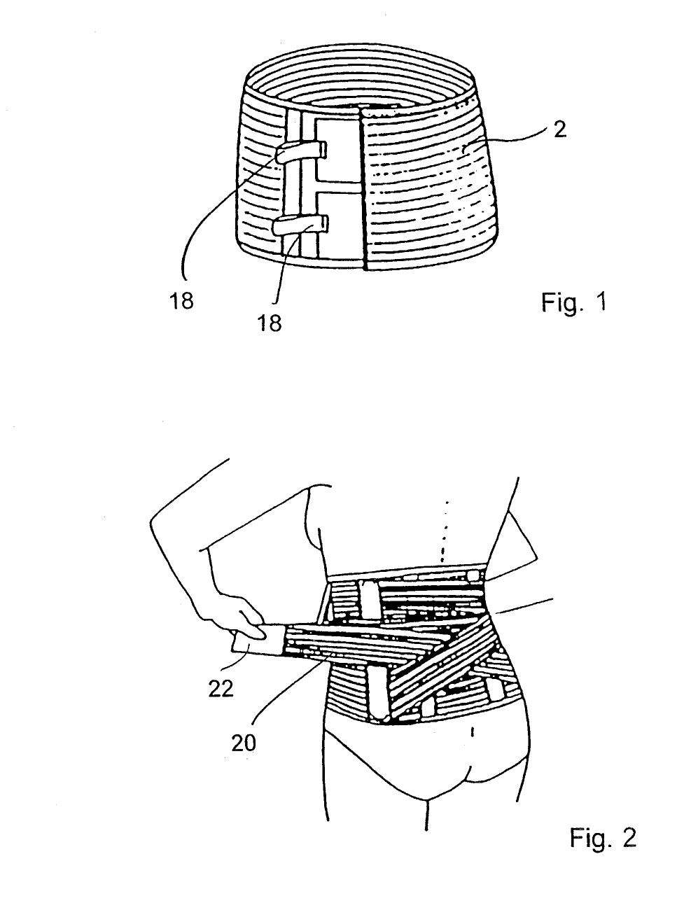

FIG. 1 shows a first embodiment of the bandage according to the invention,

FIG. 2 illustrates a second embodiment of the invention,

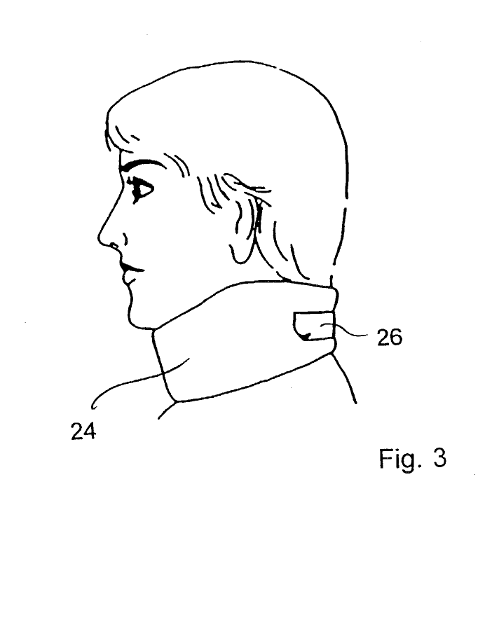

FIG. 3 shows a third embodiment of the bandage according to the invention,

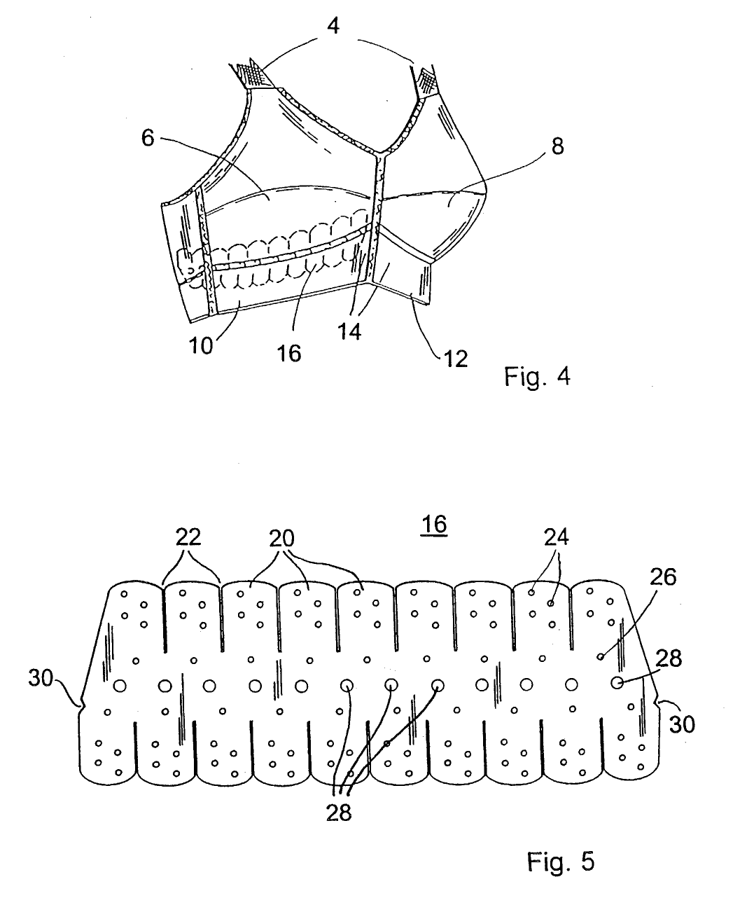

FIG. 4 shows a fourth embodiment of the invention, and

FIG. 5 is a view of the supporting means in the embodiment according to FIG. 4.

FIG. 1 shows a first embodiment of the bandage according to the invention in the form of a back brace for the lumbar and sacral regions, i.e. a lumbosacral orthosis. The bandage comprises a relatively broad, belt-like supporting means 2 made of cross-linked polythene material for application to the patient's trunk in the lumbar and sacral regions. The supporting means 2 can be covered with a soft cotton cloth to keep the supporting means from rubbing against the patient's skin and causing e.g. chafing, sticking due to sweat etc. The supporting means 2 is flexible so it can be opened and applied to the patient's body. The ends of the supporting means are then interconnected with appropriate coupling or fastening means 18.

FIG. 2 shows a second embodiment of the bandage according to the invention in the form of an alternative version of a lumbosacral orthosis. In this embodiment, the supporting means consists of a broad belt 20 made of cross-linked polythene material. The belt 20 is narrower and higher than the belt shown in FIG. 1. Bandaging is achieved by wrapping the supporting means 20 around the patient's body several times across the lumbar and sacral region. The free end of the belt 20 has an appropriate fastener 22 for attaching the belt after it has been wrapped around the patient. Even in this instance, the supporting means 20 can be covered by a soft cotton cloth for more comfortable contact with the patient's skin.

FIG. 3 shows a third embodiment of the bandage according to the invention in the form of a neck support collar, i.e. a cervical orthosis. In this instance, the supporting means 24 is covered with a textile material over some suitable intermediate padding to keep the supporting means from chafing or causing the patient any other discomfort. After the supporting means has been applied to the patient's neck, its ends are connected with suitable fasteners or attachment means 26, e.g. Velcro® closure.

The supporting means of polythene material is perforated to allow the skin to ‘breathe’ through the material, cf. the description of FIG. 5 below. The stiffness of the supporting means can be varied by varying the thickness of the material and/or the degree of perforation. In this way, different kinds of bandages, e.g. in the form of spinal orthoses, can be simply achieved. The most suitable type of bandage is governed by the prevailing disorder of the back or cervical spine. Relatively soft bandages are mainly used for acute and chronic back pain, especially in the lumbar region. Hard or stiff bandages are used for deformation of spinal column, fractures, back instability etc. These hard bandages are often custom made from plaster moulds of the body part in question, see the description below of the method according to the invention.

FIG. 4 is a frontal view of a brassiere with shoulder straps, suggested at 4, and cups 6, 8 for the breasts. The section 10, 12 under the cups 6, 8 is made of an elastic material, e.g. an elastic sold by PIAVE under the brand name TOP-TEX. The cups 6, 8 can be made of a resilient or non-resilient cloth.

The elastic section 10, 12 and the cups 6, 8 are cut so the elastic material rises to form a gap, essentially in the shape of an inverted V, in the central area 14 between the cups 6, 8. This allows unrestricted movement of the diaphragm.

An oblong, belt-like supporting means 16 is arranged in each half of the brassiere. This supporting means 16 extends from the area at the middle of the front of the brassiere to the rear section of the brassiere. The supporting means 16 can be bent along its midline, and the supporting means 16 is arranged in the brassiere in such a way that this midline parallels the lower edge of the cup 6. The supporting means 16 thereby smoothly adapts to the transition between the base of the breast and the body below when the brassiere is worn.

The supporting means 16 is devised as an elastic, flexible but relatively stiff belt of cross-linked polythene material which does not release any harmful softeners.

The cross-linked polythene material used can withstand temperatures up to 80° C. with no loss of shape and no cracking of the supporting means. This makes it possible to wash the brassiere at temperatures high enough, i.e. 60° C. or more, to kill bacteria. Better hygiene is therefore the result. The material also retains its elasticity and flexural stiffness. Its excellent heat resistance means the supporting means also retains its shape when the brassiere is washed at this relatively high temperature.

The supporting means 16 is movingly arranged between the brassiere's lining and the outer covering. The supporting means 16 therefore has some mobility, allowing it to follow the body's movements smoothly. The mobility of the supporting means 16 enables the means to support and lift the breasts in a positive fashion giving the wearer great freedom of movement.

Since the supporting means 16 extends partly to the rear across the back of the brassiere, it also supports the brassiere wearer's back, thereby contributing to better posture for the wearer. In contrast to the situation with conventional brassieres whose sole purpose is to hold the breasts at the front. The brassiere therefore prevents poor posture in the wearer, whereas conventional brassieres, whose weight is supported by shoulder straps, often make poor posture worse.

The transverse edge of the support means 16, especially the end facing the middle of the brassiere, runs at an angle across at least a part of the width of the supporting means, see also FIG. 5. The transverse edges of the supporting means 16 opposite each other at the mid-section 14 of the brassiere form a gap, essentially in the shape of an inverted V, between the supporting means 16. The supporting means 16 therefore do not obstruct the movements of the diaphragm so breathing is unimpeded. This makes the brassiere particularly suitable for use in sports, for singing etc. The supporting means 16 have no protruding corners in the mid-section 14 which could penetrate the brassiere and body fat when the wearer bends over. The design of the supporting means 16 with an angled edge 18 has been shown to be better at following the body's contours and providing better spreading of supporting means flaps 20, thereby contributing to improved support for the bust.

As FIG. 5 shows, slits 22 are arranged along the longitudinal edges of the supporting means 16. The slits 22 are perpendicular to the longitudinal axis of the supporting means and cut part way into the width of the supporting means 16, causing the formation of a number of individual, moveable flaps 20 on either side of the supporting means 16. As a result, the supporting means 16 yields and adapts to the shape of the body and bosom in a smooth fashion without any bulging. For satisfactory support of the breasts, the flaps 20 must be able to spread, something made possible by this design. This is facilitated by the fact that the section 10, 12 is made of an elastic material. If the section 10, 12 were made of a non-elastic material, the flaps 20 would be forced to stay together and therefore lack the ability to spread in adapting to the contours of the body.

The cross-linked polythene material is permeable to air, enabling the skin to ‘breathe’ through the supporting means and improving comfort for the brassiere wearer. In order to increase permeability for greater air circulation, the supporting means 16 can be perforated with ‘breathing holes’ in the flaps 24 and in the middle of the supporting means 26, 28. The ‘breathing holes’ are somewhat larger than the other perforations and arranged in a longitudinal line 28, forming a bending line for the supporting means 16. When the brassiere is worn, the supporting means 16 accordingly bends along this bending line so its upper part in FIG. 2 rests on the base of the breast while its lower half rests on the body.

It is very important for the supporting means 16 to be installed in the brassiere in the proper manner. Fasteners 30 are therefore arranged in the transverse edges of the supporting means 16 and used for mounting the supporting means in the correct position. Even a slight misplacement of the supporting means in the brassiere can greatly impair the efficacy of lifting and support.

A similar bandage can also be incorporated in garments, such as summer dresses, exercise and sports clothing, swimming suits, body stockings etc.

In fabrication of the bandage according to the invention, the polythene material, in the form of strips, slabs, sheets etc., is heated to a predetermined moulding temperature, from 90°- 180° C. At this temperature, the material acquires a consistency suitable for moulding, making it possible to fabricate a supporting means with the desired shape for the application in question. This is appropriately performed in a thermoforming machine with a ‘hot chamber’ in which the plastic is heated to the aforesaid moulding temperature and applied to a model, e.g. a plaster moulding of the body part in question. The plastic-coated model is automatically transferred to a ‘cold chamber’for rapid cooling. If a support bandage for, e.g. the forearm, is to be fabricated, a supporting means is accordingly devised which fits the shape of the forearm. After the rapid cooling, the material has an elastic flexural stiffness which it retains over time, even when used in a bandage of the kind washed a large number of times between different uses, as described above. The supporting means can be moulded closed, and then be slit longitudinally, opened along the split and applied to the arm. The elasticity of the supporting means then keeps the supporting means affixed to the arm. The bandage can also be wrapped in gauze to keep the supporting means from opening. Or the supporting means is not fabricated closed but fabricated with a longitudinal slit or groove of a suitable width to allow application as described above. In other instances, devising the supporting means with a transverse slit in the form of a sector of a size suitable for the application in question may be appropriate.

Especially in the case of hard or stiff bandages, e.g. for fixation in fractures, the supporting means can comprise two or more supporting means parts which are interconnected with suitable fastening means after being placed around the body part in question.

Or the supporting means parts can be joined with an elastic material making it possible to apply the bandage by pulling it over an arm or leg or up to the desired part of the back etc. by temporarily stretching the bandage. The material's elasticity then retracts the bandage after it has been applied to the desired site.

The material used for fabricating the supporting means can be pre-perforated to increase the passage of air through the supporting means. The polythene mesh material has an ability to retain the perforations after the material is heated to the moulding temperature and shaped into the desired supporting means. Perforation can be uniform across the material or varied in some suitable fashion, e.g. with perforations more closely spaced or concentrated to certain areas of the material and the fabricated supporting means.

1. A bandage for orthopaedic use comprising an elastic, flexible relatively stiff supporting means of a cross-linked polyethylene material, tailored to the contours of a body part to be supported, characterized in that the polythene material includes a silane additive with at least one hydrolysable organic group with a high degree of compatibility with the polythene composition.

2. The bandage according to claim 1, characterized in that the supporting means is perforated.

3. The bandage according to claim 1 or 2, characterized in that the stiffness of the supporting means can be varied by varying the thickness of the material and/or the degree of perforation.

4. The bandage according to any of the previous claims, characterized in that the bandage is a brace with the supporting means intended to essentially enclose the brace wearer's trunk, or part thereof.

5. The bandage according to any of claims 1-3, characterized in that the supporting means is devised to follow the contours of and largely enclose an extremity of the bandage wearer, thereby forming a support bandage for the said extremity.

6. The bandage according to any of the previous claims, characterized in that the supporting means comprises at least two supporting means parts devised for application to the body part to be supported, and in that the supporting means parts are interconnectable.

7. The bandage according to claim 6, characterized in that the said supporting means parts are interconnectable with an elastic material.

8. The bandage according to claim 6, characterized in that the said supporting means parts are interconnectable with an external bandage made of a non-elastic material.

9. The bandage according to any of claims 1-3, characterized in that the supporting means is belt-like and devised for insertion into each half of a brassiere or garment with a brassiere section including brassiere cups, such as exercise suits and sports clothing, bathing suits, body stockings etc., covering the area from the middle of the front section of the brassiere or garment to the rear section of the brassiere or garment, the supporting means being bendable along a longitudinal line, mainly extending along the lower edge of the brassiere or garment half, the part of the belt-like supporting means on one side of said line resting on the base of the breast when the brassiere or garment is worn, with support provided by the other part which rests against the body, under the breast, of the brassiere or garment wearer.

10. The bandage according to claim 9, characterized in that transverse slits are arranged in the longitudinal edges of the belt-like supporting means to enable the supporting means to follow the contours of the bust and body smoothly.

11. The bandage according to claim 9 or 10, characterized in that the transverse edges of the supporting means facing the mid-section of the brassiere or garment run at an angle across at least a part of the width of the supporting means in such a way that the transverse edges of the supporting means, facing the brassiere's mid-section, form a gap between the supporting means in the basic shape of an inverted V.

12. The bandage according to claim 10 or 11, characterized in that the transverse slits are arranged in a zigzag pattern along the longitudinal edges of the supporting means.

13. The bandage according to any of claims 9-12, characterized in that a row of perforation holes, larger than other perforation holes, are arranged along said longitudinal line between the supporting means parts, for support to the base of the breasts and to the body respectively, to form a bending line for the supporting means.

14. The bandage according to any of claims 9-13, characterized in that the supporting means are located between the lining and the outer covering of the brassiere or garment.

15. The bandage according to any of claims 9-14, characterized in that the transverse edges of the supporting means are equipped with means to ensure correct mounting in the brassiere or garment.

16. The bandage according to any of the previous claims, characterized in that the supporting means is devised even to protect a body part against blows, impacts etc.

17. A method for fabricating a bandage according to any of the previous claims, characterized in that the polythene material is heated to a predetermined moulding temperature, at which the material has a suitable consistency for moulding, supporting means with the desired shape then being made from the material.

18. The method according to claim 17, characterized in that the said moulding temperature ranges from about 90° to 180° C.

19. The method according to claim 17 or 18, characterized in that the polythene material is perforated before being heated to the moulding temperature.
